(19) United States
(12) Patent Application Publication
(10) Pub. No.: US 2010/0292327 A1
(43) Pub. Date: Nov. 18, 2010

(54) TREATMENT OF ASPIRIN RESISTANCE WITH BETAINE AND/OR BETAINE ENRICHED MOLASSES

(76) Inventor: Jallal Messadek, Liege (BE)

Correspondence Address: Hovey Williams LLP, 10801 Mastin Blvd., Suite 1000, Overland Park, KS, 66210, US

(21) Appl. No.: 12/783,377

(22) Filed: May 19, 2010

(30) Foreign Application Priority Data
Nov. 21, 2007 (BE) PCT/BE2007/000121
Nov. 21, 2008 (BE) PCT/BE2008/000096

(51) Int. Cl. A61K 31/205; A61P 9/00; A23L 1/30; A23L 1/305; A23L 3/44; A23L 3/46; A23L 3/40; A23P 1/06; A23L 1/09

(52) U.S. Cl. 514/551; 426/648; 426/490; 426/385; 426/471; 426/465; 426/473

(57) ABSTRACT

Use of glycine betaine as therapeutically active ingredient for the preparation of a medicament and/or a nutritional product and/or a dietary supplement for treating human resistant to a compound selected from the group consisting of aspirin, clopidogrel, thienopyridines and combinations thereof, who suffers from a disease requiring the administration of a compound selected from the group consisting of aspirin, clopidogrel, thienopyridines and combinations thereof, or who is at risk of suffering of said disease, whereby the medicament comprises an effective amount of glycine betaine for decreasing by at least 10%, advantageously at least 20% the resistance of said patient to said compound selected from the group consisting of aspirin, clopidogrel, thienopyridines and combinations thereof

### CROSS-REFERENCE TO RELATED APPLICATIONS

The present application is a continuation-in-part of PCT/BE2008/000096, filed on Nov. 21, 2008, and published as WO 2009/065193, which claims the priority benefit under the Paris Convention of PCT/BE2007/000121, filed on Nov. 21, 2007, the contents of which are incorporated by reference herein in their entirety.

### FIELD OF THE INVENTION

The present invention relates a betaine, preferably glycine betaine of formula (CH₃)₃N⁺(CH₂) COO⁻, and/or a betaine enriched and/or betaine concentrated molasses for the fabrication of a medicament and/or nutritional product. In particular, the present invention relates to the use of such medicament and/or nutritional product for treatment and/or the prevention of aspirin resistant cardio-cerebrovascular diseases using betaine and/or sugar beet and/or sugar cane molasses enriched in betaine theirs natural extracts, formulations and compositions comprising any or both of them.

Surprisingly it was found that patients who are aspirin and/or clopidogrel resistant and/or thienopyridines resistant can beneficiate of the betaine based medicaments and/or nutritional products for treatment and/or the prevention of aspirin and/or clopidogrel and/or thienopyridines resistances.

Indeed the pharmaceutical betaines, preferably glycine betaine of formula (CH₃)₃N⁺(CH₂) COO⁻, and/or betaine enriched molasses nutritional products according to the present invention, when administrated to patients in need, i.e. experiencing aspirin and/or clopidogrel and/or thienopyridines resistance, overcome and/or control and/or treat and/or prevent these resistances and/or non-responses and/or semi-responses.

### BACKGROUND OF THE INVENTION

Cardiovascular disease ranks as a leading cause of mortality and morbidity and represents a significant drain on health resources in many countries.

It is well established that aspirin therapy reduces the risk of a stroke and a first heart attack in healthy individuals, and subsequent heart attacks, strokes, or cardiovascular death in patients with established cardiovascular disease. For example, U.S. Pat. No. 5,240,917 relates to percutaneous administration of aspirin as antithrombotic agent. Studies have shown that aspirin reduces the risk of cardiovascular events by as much as 25% in patients with arterial vascular disease.

Most heart attacks and strokes are caused by blood clots in the heart or brain arteries that form on top of cracked atherosclerotic plaques. These blood clots are predominantly composed of clumped platelets. Aspirin works to prevent blood clot formation at these sites by reducing the ability of the platelets to clump together and form platelet aggregates. Aspirin, also known as acetylsalicylic acid, reduces platelet reactivity because its acetyl group acetylates a key intra-platelet enzyme known as cyclo-oxygenase. Once acetylated, cyclo-oxygenase cannot work to generate thromboxane A2, a substance released from the platelets that serves to activate other platelets and induce them to clump together in aggregates. In order for aspirin to work, therefore, it must reduce thromboxane A2 levels.

Thromboxane A2 has a very short half-life, and is rapidly converted to a stable metabolite called thromboxane B2. Although thromboxane B2 can be measured in blood, the tests can be problematic because platelets can be activated during the collection process. Once activated, the platelets will release thromboxanes that can interfere with the assay. It is therefore preferable to measure thromboxane B2 in the urine.

Aspirin is effective for patients with heart attacks, strokes or peripheral arterial disease or those at risk of these disorders. A role for aspirin in reducing the risk of fatal colon cancer has also been suggested and aspirin may be useful in the treatment of patients with antiphospholipid antibodies, including the lupus anticoagulant. Thus, determining the effectiveness of aspirin treatment in many conditions is an important prognostic factor and may help physicians recommend the most appropriate therapeutic course.

While aspirin is effective in many individuals, approximately 10 to 20% of patients with arterial thrombosis who are treated with aspirin have a recurrent vascular event during long-term follow-up.

The failure of these patients to derive a beneficial effect from aspirin is termed “aspirin resistance”. There are several possible explanations for aspirin resistance but, whatever the underlying cause, the result is the same: the likelihood of onset of a cardiovascular and/or a cerebrovascular event.

### SUMMARY OF THE INVENTION

Those people at particular risk of having a recurrent vascular event can be identified, so that they can be appropriately treated before a heart attack or stroke occurs, by using a rapid test for determining aspirin resistance such as the urinary levels of thromboxane A2 levels or whole blood aggregometry. Aspirin resistance, defined as failure of suppression of thromboxane generation, and cardiovascular risk. Determination of the degree of resistance to aspirin is used to predict the risk of a cardiovascular event or other condition that would benefit from lowering thromboxane A2 levels. In the invention, treatment with pharmaceutical betaines, preferably glycine betaine, and/or betaine enriched molasses nutritional products can decrease aspirin resistance by at least 10%, advantageously by at least 20%, preferably by at least 30%, and more preferably by at least 50%.

Urinary thromboxane A2 metabolite levels in patients are an accurate predictor of recurrent cardiovascular mortality. Thus, determination of metabolite levels in patients may serve to identify those patients at particular risk of developing cardiac ischemia or stroke and accordingly the patient population which can beneficiate of the treatments provided by the present invention namely pharmaceutical betaines, preferably glycine betaine, and/or betaine enriched molasses nutritional products.

In one aspect of the invention, the method for assessing aspirin resistance in a patient comprises determining the concentration of a metabolite of thromboxane A2 in a sample of body fluid from the patient. The method preferably further comprises the step of comparing the concentration of metabolite in the sample to a predetermined set of concentration quartiles to determine within which quartile the sample falls and determining aspirin resistance based on the quartile of the sample. A concentration of metabolite within the second, third or fourth quartile is indicative of an increased risk of a cardiovascular event.

In another aspect, the method of screening a patient for risk of having a cardiovascular event comprises contacting a body fluid sample from the patient with an antibody which specifically binds to a thromboxane-A2 metabolite, determining the degree of immune complex formation by immunoassay, and assessing the patient's risk of a cardiovascular event upon the basis of immune complex formation.

In a preferred embodiment, the patient has arterial vascular disease and/or risk of a recurrent vascular event.

In a further preferred embodiment, the metabolite that is measured is thromboxane-B2 metabolite, preferably 11-dehydro thromboxane B2.

In a further aspect, a urine level of this metabolite of greater than 15 ng/mmol creatinine is indicative of risk of a cardiovascular event, more preferably a urine level greater than 21.9 ng/mmol creatinine is indicative of risk of a cardiovascular event and most preferably a urine level greater than 33.8 ng/mmol creatinine is indicative of risk of a cardiovascular event. In the invention, administering pharmaceutical betaines, preferably glycine betaine, and/or betaine enriched molasses nutritional products to the patient decreases the high levels of urinary and/or blood levels of the metabolit by at least 10%, advantageously by at least 20%, preferably by at least 30%, and more preferably by at least 50%.

A useful kit for determining aspirin poor responder according to the present application is the Cayman Chemical kit reference 10010153. Aspirin Effect-Detection Kit is a 510K, clinically approved diagnostic kit for the measurement of 11-dehydro TXB₂

In another aspect of the invention whole blood aggregometry is used to determine aspirin resistance and/or clopidogrel and/or thienopyridines resistance in a patient in need. Aspirin and/or clopidogrel and/or thienopyridines resistance can be defined then as an aggregation inhibition less than 10% reduction in response to activation by ADP or activation by Arachidonic Acid or activation by Collagen compared with pretreatment values.

According to the invention the threshold concentration for agonist to be used in platelet and/or whole blood aggregation, is 1 μg/ml for collagen and 0.5 mM for arachidonic acid.

According to the invention the levels of platelet or blood aggregation of a human aspirin resistant are inferior by less than 20% comparatively to the levels of platelet or blood aggregation of said human prior being administered one or more antiplatelet compounds selected from the group consisting of aspirin, aspirin derivatives and combinations thereof, when using agonists at concentration of 1 μg/ml for collagen and 0.5 mM for arachidonic acid.

### BACKGROUND

Aspirin belongs to a class of medicines known as non-steroidal anti-inflammatory drugs (NSAIDs). It is an effective anti-inflammatory drug with both analgesic and antipyretic effects. Ti works by blocking the production of prostaglandin. The most commonly known side effects of aspirin include: Gastrointestinal reactions, such as poor appetite, peptic ulcers, and in some cases, even perforation, Allergic reaction Acute renal failure and chronic interstitial nephritis, etc.

As researches become more and more extensive, aspirin's indications have been enlarged to the treatment and secondary prevention of cerebral arteriosclerosis, coronary heart disease, and myocardial infarction.

The Food and Drug Administration has recently recommended that persons suffering from thrombotic disease take from between 50 mg aspirin/day to 325 mg aspirin/day. However, higher doses of aspirin pose health risks in certain segments of the population, including stomach irritation, ringing in ears, allergic reactions and in children, Reye's syndrome.

Platelets can be activated by pathways not blocked by aspirin, such as ADP. This well-recognized fact may contribute to the 8% to 45% of the population who are, in vitro, aspirin resistant. Researchers propose that platelets from aspirin-resistant patients appear to be hypersensitive to ADP, which may explain aspirin resistance and may justify therapeutic improvement through the use of alternative antiplatelet agents.

Although aspirin inhibits platelet activation and reduces atherothrombotic complications in patients at risk for MI and stroke, some patients are aspirin resistant, which means that a sufficient inhibition of platelet function by aspirin is not always achieved. In fact, patients undergoing coronary artery bypass grafting (CABG) are known to have a high incidence of aspirin resistance. One prospective study determined the prevalence and clinical predictors of aspirin resistance in 325 patients with stable cardiovascular disease who were receiving aspirin (325 mg/day for seven or more days) but no other antiplatelet agents. The widely used optical-platelet aggregation method and the newer, quicker, platelet-function-analyzer 100 method, a whole blood test measuring platelet adhesion and aggregation ex vivo, were employed. Researchers found patients who were either aspirin resistant or aspirin semiresponders were more likely women and less likely smokers compared with aspirin-sensitive patients. A trend toward increased age was also discovered in aspirin-resistant patients or aspirin semiresponders, while there were no differences in aspirin sensitivity by platelet count, race, diabetes, renal disease, or liver disease. Overall, up to 10% of patients are resistant to aspirin and have higher rates of cardiovascular events. These are the patients who may specifically benefit from the present invention therapies.

At present, aspirin is widely used in the treatment of cardiovascular disease. The mechanism is that aspirin can block the production of thromboxane A2 (TXA2) in vivo. As TXA2 can promote platelet conglutination and coagulation, aspirin can reduce the incidences of arteriosclerosis and myocardial infarction by inhibiting platelet aggregation. But researches have found that the biosynthesis of thromboxane A2 is not effectively prevented in some patients after they take the drug. That is, aspirin looses its protective effects on cerebrovascular and cardiovascular systems.

This is called aspirin resistance (AR). For most patients, aspirin can reduce the risk of cardiovascular disease by 25%, but for patients with aspirin resistance, administration of aspirin, instead of protecting from cardiovascular events, can increase the incidence of myocardial infarction and stroke. These findings have limited the application of aspirin.

Yusuf S etc. reported that, in patients with acute coronary syndrome receiving aspirin, including those undergoing percutaneous coronary intervention, administration of clopidogrel in addition to treatment with aspirin is beneficial in reducing the incidence of early and long-term major cardiovascular events. [“Effects of pretreatment with clopidogrel and aspirin followed by long-term therapy in patients undergoing percutaneous coronary intervention: the PCI-CURE study”. Lancet, 2001].

At present, for researches on aspirin resistance, immunoenzyme assay is commonly used for the measurement of the level of urinary 1′-dehydrothromboxane B.sub.2 (TXB.sub.2) in the urine sample of patients before taking drug. 11-dehydrothromboxane B.sub.2 is a metabolite of thromboxane A.sub.2. High urinary 11-dehydrothromboxane B.sub.2 level can identify patients with aspirin resistance and drugs having effects on easing aspirin resistance.

### Clopidogrel Resistance

More recently, a similar variable platelet response and potential resistance to therapy has emerged with thienopyridines. Studies have shown a dose- and time-dependent variability in response to clopidogrel as measured by optical platelet aggregometry in response to ADP. In a study by Gurbel et al, (Clopidogrel for coronary stenting: response variability, drug resistance, and the effect of pretreatment platelet reactivity. Circulation. 2003; 107: 2908-2913.) 96 patients undergoing elective coronary stenting were monitored before and at multiple time points after standard clopidogrel therapy (300-mg loading dose followed by 75 mg daily). Clopidogrel resistance, empirically defined as <10% reduction in aggregation in response to 5 μmol/L ADP compared with pretreatment values, was seen in 63% of patients at 2 hours, 31% at 24 hours, 31% at 5 days, and 15% at 30 days. Patients with the highest pretreatment values had the least antithrombotic protection over the first 5 days. In another report, Muller et al (Prevalence of clopidogrel non-responders among patients with stable angina pectoris scheduled for elective coronary stent placement. Thromb Haemost. 2003; 89: 783-787) defined nonresponders as those with <10% reduction in platelet aggregation to ADP and semiresponders as those with 10% to 29% reduction 4 hours after 600-mg clopidogrel load, as no additional effect was seen with this treatment regimen at 24 hours. This study found that to 5 μmol/L ADP, 5% were nonresponders and 9% were semiresponders, and to 20 μmol/L ADP, 11% were nonresponders and 26% were semiresponders. Although not designed to evaluate clinical outcomes, an intriguing finding in the Muller study was that 2 patients (of 105 tested) developed subacute stent thrombosis, and both met the definition of clopidogrel nonresponse. An additional report correlated anginal class to platelet inhibition and found that patients with higher anginal class on presentation had less inhibition of platelet aggregation after loading with 450 mg of ADP.

Indeed the pharmaceutical betaines and betaine enriched molasses nutritional products of the present invention and according to the present invention, when administrated to patients in need, i.e. experiencing clopidogrel and/or thienopyridines resistance, overcome and/or control and/or treat and/or prevent these resistances and/or nonresponses and/or semiresponses.

One aspect of the present invention relates to the use of Betaine and its extract in the treatment of aspirin resistant cardiovascular and cerebrovascular diseases.

The purpose of the present invention is to provide the use a betaine, preferably glycine betaine of formula (CH₃)₃N⁺(CH₂) COO⁻, and/or a betaine enriched molasse for the fabrication of a medicament and/or nutritional product for treating aspirin resistance.

Said aspirin resistance refers to an inability to effectively inhibit the biosynthesis of thromboxane A.sub.2 after taking aspirin. That is, aspirin loses its protective effect on cardiovascular and cerebrovascular system. In the majority of patients, aspirin can reduce the risk of cardiovascular and cerebrovascular diseases by 25%. But for patients with aspirin resistance, treatment of cardiovascular and cerebrovascular diseases with aspirin can not prevent them from the cardiovascular and cerebrovascular events, but instead, can increase the risk of myocardial infarction and stroke.

In the present invention these cardiovascular and cerebrovascular diseases are called aspirin resistant cardiovascular and cerebrovascular diseases, particularly coronary heart disease and angina pectoris in which aspirin treatment is ineffective. In this invention, drugs that are effective in the treatment of aspirin resistant cardiovascular and cerebrovascular diseases are called drugs of anti-aspirin resistance; this action is called effect of anti-aspirin resistance.

The present invention adopts a now commonly used method for research of aspirin resistance. It uses immunoenzyme assay to test the urinary sample of the patients and analyze the change in the level of urinary 11-dehydrothromboxane B.sub.2 (TXB.sub.2) to determine if there is any reduction of aspirin resistance in the patients after taking Betaine preparations. From clinical investigations, the present invention confirms that Betaine and its extract have effects of reducing aspirin resistance, and can be used as drugs of anti-aspirin resistance and for the preparation of drugs of anti-aspirin resistance.

Another aspect of the present invention relates to the use of a composition that contains Betaine as active ingredient in the treatment of aspirin resistant cardiovascular and cerebrovascular diseases.

Another aspect of the present invention relates to the use of a betaine, preferably glycine betaine of formula (CH₃)₃N⁺(CH₂) COO⁻, and/or a betaine enriched molasse for the fabrication of a medicament and/or nutritional product or any pharmaceutical preparation. The preferred oral preparations are drop pills, spray solutions, pellets, pills, granules, capsules, tablets, powders, gel soil capsule(s) and oral liquids.

### DETAILED DESCRIPTION OF THE INVENTION

In the field of the present invention betaines, preferably glycine betaine can be used in food grade and/or in pharmaceutical grade for achieving the goals of the invention.

In the field of the present invention betaine comes and is extracted from natural sources such as sugar beet, sugar cane, spinach or cereals and theirs natural extracts. Optionally, it can also be synthesized by chemical means.

In one embodiment, the betaines of the invention can be extracted from vinasses and/or dry matter resulting from bioethanol production.

The pharmaceutical and/or nutritional products in the scope of the present invention shall provide to patients in need effective oral daily doses between 5 mg/kg and 200 mg/kg, preferably between 10 mg/kg and 100 mg/kg more preferably between 15 mg/kg and 50 mg/kg.

Beet and/or cane molasses after desugarization processes are further submitted to one or more separation processes as to obtain betaine enriched molasses the fabrication of a nutritional product wherein said molasse comprises between 10 and 95% betaine, more specifically 10% and 90% of betaine as dry matter basis, advantageously between 20% and 80%, preferably between 30% and 70% more preferably between 40% and 60%.

Such betaine enriched molasses are possibly submitted to one or more drying processes selected from the group consisting of drying processes, spray-drying, tumble-drying, lyophilization processes, freeze-drying processes and their mixtures.

Such betaine enriched molasses after being dried are possibly submitted to one or more selected from the group consisting of powdering, micronizing, grinding, crushing, granulating and their mixtures. The obtained grinded and/or powdered dry products having sizes preferably between 10 and 200 meshes and can be included in different edible products for human use.

In one embodiment the obtained grinded and/or powdered dry products having sizes preferably between 10 and 200 meshes are packaged in unit oral dosage forms such as capsules drop pills, spray solutions, pellets, pills, granules, capsules, tablets, powders and oral liquids.

In one embodiment the obtained grinded and/or powdered dry products having sizes preferably between 10 and 200 meshes are packaged in unit dosage forms selected from the group consisting of sachets, pouches, blisters and bags, wherein the pharmaceutical unit dosage form is provided with moisture harrier property defined by an MVTR value inferior to 0.2 g/m², advantageously inferior to 0.1 g/m², preferably inferior to 0.01 g/m², more preferably inferior to 0.001 g/m², specifically inferior to 0.0001 g/m² at a temperature of 38° C. and at 90% relative humidity during 24 hours. In one embodiment the molasses after being betaine enriched are used in the fabrication of nutritional products for human use.

In one embodiment the molasses after being betaine enriched are used in the fabrication of oral unit dosage forms selected from the group consisting of drop pills, spray solutions, pellets, pills, granules, capsules, tablets, powders and oral liquids.

In one embodiment the molasses after being betaine enriched are packaged in oral unit dosage forms selected from the group consisting of sachets, pouches, blisters and bags, wherein the pharmaceutical unit dosage form is provided with moisture barrier property defined by an MVTR (Moisture Vapor Transmission Rate) value inferior to 0.2 g/m², advantageously inferior to 0.1 g/m², preferably inferior to 0.01 g/m², more preferably inferior to 0.001 g/m², specifically inferior to 0.0001 g/m² at a temperature of 38° C. and at 90% relative humidity during 24 hours.

As used herein, the terms “molasses” and “molasse” refer to the dark syrup which is left behind after the bulk sugar crystals are collected in the sugar beet/cane mill, the black syrup remaining after the sugar beet/cane syrup has been centrifuged for the last time in the refinery or beet molasses.

Sugar beet molasses after sugar extraction process usually have 5% betaine content. The molasses extract may contain one or more of the following substances: nitrates, lipids, phospholipids, proteins, amino acids, flavonoids such as anthocyanins, catechins, chalcones, flavonols and flavones, polyphenols, antioxidants, phytosterols such as 1-octacosanol, campesterol, stigmasterol, p-sitosterol, oligosaccharides such as raffinose, 1-kestose, theanderose, 6-kestose, panose, neo-kestose and nystose, and organic acids such as c-aconitic acid, citric acid, phosphoric acid, gluconic acid, malic acid, t-aconitic acid, succinic acid and lactic acid, aliphatic alcohols, vitamins, minerals, carbohydrates, gums and neutral and polar lipids.

The sugar cane or sugar beet field trash/fibrated sugar cane tops extract may contain one or more of the following substances: lipids, phospholipids, amino acids, proteins, flavonoids such as anthocyanins, catechins, chalcones, flavonols and flavones, polyphenols, antioxidants, phytosterols such as 1-octacosanol, campesterol, stigmasterol, P-sitosterol, oligosaccharides such as raffinose, 1-kestose, theanderose, 6-kestose, panose, neo-kestose, and nystose, and organic acids such as c-aconitic acid, citric acid, phosphoric acid, gluconic acid, malic acid, t-aconitic acid, succinic acid and lactic acid, aliphatic alcohols, vitamins, minerals, carbohydrates, gums and neutral and polar lipids.

The bagasse/pulp extract may contain one or more of the following substances: lipids, phospholipids, amino acids, proteins, flavonoids such as anthocyanins, catechins, chalcones, flavonols and flavones, polyphenols, antioxidants, phytosterols such as 1-octacosanol, campesterol, stigmasterol, p-sitosterol, oligosaccharides such as raffinose, 1-kestose, theanderose, 6-kestose, panose, neo-kestose and nystose, and organic acids such as c-aconitic acid, citric acid, phosphoric acid, gluconic acid, malic acid, t-aconitic acid, succinic acid and lactic acid, aliphatic alcohols, vitamins, minerals, carbohydrates, gums and neutral and polar lipids.

Preferably, the molasses and/or vinasses used are from the sugar beet. Pure fractions of components, preferably betaines, are recovered and can be concentrated by chromatographic separations, microfiltrations, reverse osmosis, vacuum evaporations, crystallizations, dryings and freeze dryings.

As used herein, the terms “nutritional” and/or “nutritional product(s)” and/or “supplement product(s)” and/or “dietary supplement” and/or “food product(s)” include any edible product, such as but not limited to confectioneries, supplements, snacks (sweet and savoury), cocoa-containing foods, flavours, beverages, dietary supplements and formulations including supplements used in animal health and nutrition. Confectioneries refer to any sweetened foods, including but not limited to candy, chocolate, chewing gum, icings, fruit pulp based delivery systems and the like. Additional ingredients desired in the resulting food product may be added at any point in the process. Food products may also encompass for example, complex confections where chocolate is combined with and generally coats other foods such as caramels, nougat, fruit pieces, nuts, wafers, biscuits, ice cream or the like.

In one embodiment, the compounds and/or the medical products and/or the nutritional products of the invention may be extracted from red beet and/or quinoa.

The betaine enriched molasses and/or food or nutritional products also may contain sucrose or other sugars. The betaine enriched molasses can contain from about 5 to about 65% by weight sugar. Preferably, the food or nutritional products contain no more than about 25% by weight sugars, based upon the total weight of the products, with products containing no more than about 20% by weight sugars being preferred. Products containing no more than about 15% by weight sugars are more preferred, with products containing no more than about 10% sugars being even more preferred. Products containing no more than about 5% sugars are the most preferred.

The betaine enriched molasses and/or food or nutritional products also may contain organic acids such as one or more lactic acid, malic acid, acetic acid, oxalic acid, glutammic acid, citric acid, succinic acid and theirs mixtures. The betaine enriched molasses can contain from 1 to about 40% by weight organic acids. Preferably, the food or nutritional products contain no more than about 25% by weight organic acids, based upon the total weight of the products, with products containing no more than about 20% by weight organic acids being preferred. Products containing no more than about 15% by weight organic acids are more preferred, with products containing no more than about 10% organic acids being even more preferred. Products containing no more than about 5% organic acids are the most preferred.

The betaine enriched molasses and/or food or nutritional products and/or dietary supplements also may contain non essential aminoacids such as one or more lysine, methionine, cystine, tryptophan, threonine and theirs mixtures. The betaine enriched molasses can contain from about 1 to about 40% by weight essential amino acids. The betaine enriched molasses can contain from about 1 to about 30% by weight nitrates. Preferably, the food or nutritional products contain no more than about 25% by weight aminoacids, based upon the total weight of the products, with products containing no more than about 20% by weight aminoacids being preferred. Products containing no more than about 15% by weight aminoacids are more preferred, with products containing no more than about 10% aminoacids being even more preferred. Products containing no more than about 5% aminoacids are the most preferred.

The betaine enriched molasses and/or natural nutritional products and/or dietary supplements formed according to the invention can be used alone, in combination or added into foods to improve and/or to fortify the functional benefits associated with such foods. Beverages, confectionaries, dairy products, yogurts, creams, ice-creams, cookies, chocolates, and generally edible products for human use can be admixed and/or supplemented with the products of the invention.

In another embodiment according to the invention one or more vitamins can be added to the betaine enriched molasses and/or food or nutritional products and/or dietary supplements.

In another embodiment according to the invention one or more omega fatty acids can be added to the betaine enriched molasses and/or food or nutritional products and/or dietary supplements.

In another embodiment according to the invention one or more polyphenols can be added to the betaine enriched molasses and/or food or nutritional products and/or dietary supplements. In another embodiment according to the invention one or more flavanoids (or bioflavonoids) can be added to the betaine enriched molasses and/or food or nutritional products and/or dietary supplements.

According to the invention the pharmaceutical betaines, preferably glycine betaine of formula (CH₃)₃N⁺(CH₂) COO⁻, and/or betaine enriched molasses nutritional products and/or supplement products and/or food products can be used, in a patient in need, for reaching and/or achieving one or more health and/or therapeutical purpose selected from the group consisting of: improve athletic performance, improve haemostasis, sustain healthy haemostasis, favour healthy haemostasis, improve favour haemostasis, sustain haemostasis, ameliorate haemostasis, reinforce haemostasis, improve cardiovascular function, sustain healthy cardiovascular function, favour healthy cardiovascular function, favour cardiovascular function, sustain cardiovascular function, ameliorate cardiovascular function, reinforce cardiovascular function, improve blood circulation, favour healthy blood circulation, favour blood circulation, sustain healthy blood circulation, sustain blood circulation, ameliorate blood circulation, reinforce blood circulation, improve blood microcirculation, favour blood microcirculation, sustain healthy blood microcirculation, sustain blood microcirculation, ameliorate blood microcirculation, reinforce blood microcirculation, improve platelet function, improve healthy platelet function, favour healthy platelet function, favour platelet function, sustain healthy platelet function, sustain platelet function, ameliorate platelet function, reinforce platelet function, pacify platelet, favour platelet pacification, sustain platelet pacification, reinforce platelet pacification favour healthy sensitivity to aspirin, improve sensitivity to aspirin, favour sensitivity to aspirin, sustain healthy sensitivity to aspirin, sustain sensitivity to aspirin, ameliorate sensitivity to aspirin, reinforce sensitivity to aspirin, favour healthy sensitivity to clopidogrel, improve sensitivity to clopidogrel, favour sensitivity to clopidogrel, sustain healthy sensitivity to clopidogrel, improve sensitivity to clopidogrel, sustain sensitivity to clopidogrel, ameliorate sensitivity to clopidogrel, reinforce sensitivity to clopidogrel, favour healthy sensitivity to thienopyridines, improve sensitivity to thienopyridines, favour sensitivity to thienopyridines, sustain healthy sensitivity to thienopyridines, improve sensitivity to thienopyridines, sustain sensitivity to thienopyridines, ameliorate sensitivity to thienopyridines, reinforce sensitivity to thienopyridines, reduce the risk of aspirin resistance, reduce the risk of clopidogrel resistance, reduce the risk of thienopyridines resistance, ameliorate aspirin resistance, ameliorate clopidogrel resistance, ameliorate thienopyridines resistance, reduce the risk of diabetes, ameliorate diabetes, improve glycemia in diabetic states, ameliorate glycemia in diabetic states, ameliorate insulin resistance in diabetic states, favour healthy insulin sensitivity, improve insulin sensitivity, favour insulin sensitivity, sustain healthy insulin sensitivity, sustain insulin sensitivity, ameliorate insulin sensitivity, reinforce insulin sensitivity, favour healthy glycemia, favour normal glycemia, sustain healthy glycemia, sustain normal glycemia, ameliorate glycemia, reinforce healthy glycemia, reduce the risk of high glycemia, improve glucose utilisation, ameliorate glucose utilisation, ameliorate glucose utilisation by the body, favour healthy legs circulation, favour legs circulation, sustain healthy legs circulation, improve legs circulation, sustain legs circulation, ameliorate legs circulation, reinforce legs circulation, favour healthy erectile function, improve erectile function, favour erectile function, sustain healthy erectile function, sustain erectile function, ameliorate erectile function, reinforce erectile function, relieve the haemorrhoids crisis, relieve heavy legs, reduce the risk of inflammation, reduce the risk of cancer, reduce the risk of Alzheimer Disease, ameliorate vision; and the combinations of such health purposes.

The following examples are given to illustrate some aspects of the invention and can no way limit the invention.

### Example 1

Protocol Blood of 24 CAD patients on Aspirin or Aspirin+Clopidogrel is supplemented with saline or Betaine (50 μg/ml) and tested for Whole Blood Aggregometry using a Multiplate® (Dynabyte Informations System GmbH, Munich) whole blood aggregometer different agonists where used i.e.:

ASPI test: Activation by arachidonic acid to detect Aspirin effects. COL test: Collagen activates the collagen receptor, which leads to a release of endogenous arachidonic acid, which is converted to TXA2 and activates the platelet. ADP test: ADP stimulates platelet activation by the ADP receptors. The most important ADP receptor (P2Y12) is blocked by clopidogrel, prasugrel and ticlopidine. TRAP test: TRAP-6 stimulates the thrombin receptor on the platelet surface. TRAP test allows detecting GPIIb/IIIa antagonists.

The brochure of Multiplate® is incorporated in the present specification by reference.

Patients # 1, 5, 11, 12, 14, 18, 19, 20, 22, 23, 24 were on aspirin 75 mg/day or 150 mg/day. Patients # 2, 3, 4, 6, 7, 8, 9, 10, 13, 15, 16, 17, 71 were on aspirin 75 mg/day or 150 mg/day+clopidogrel 75 mg/day

Patients' blood supplemented with saline

| Patient ID | AspiTest | Collagen | ADP | TRAP |
|---|---|---|---|---|
| # 1 | 7 | 10 | 32 | 76 |
| # 2 | 6 | 11 | 18 | 67 |
| # 3 | 19 | 14 | 8 | 17 |
| # 4 | 119 | 71 | 80 | 119 |
| # 5 | 113 | 118 | 119 | 173 |
| # 6 | 116 | 77 | 86 | 104 |
| # 7 | 109 | 124 | 110 | 136 |
| # 8 | 64 | 53 | 92 | 106 |
| # 9 | 34 | 11 | 59 | 83 |
| # 10 | 37 | 18 | 8 | * |
| # 11 | 52 | 57 | 87 | 118 |
| # 12 | 22 | 20 | 57 | 89 |
| # 13 | 100 | 79 | 71 | 104 |
| # 14 | 119 | 96 | 88 | 122 |
| # 15 | 31 | 42 | 107 | 103 |
| # 16 | 31 | 26 | 57 | 94 |
| # 17 | 32 | 29 | * | 46 |
| # 18 | 80 | 52 | 87 | 121 |
| # 19 | 31 | 84 | 107 | 156 |
| # 20 | 11 | 38 | 93 | 132 |
| # 21 | 122 | 49 | 59 | 158 |
| # 22 | 150 | 37 | 143 | 176 |
| # 23 | 13 | 6 | 8 | 60 |
| # 24 | 83 | 41 | 75 | 124 |

* not realised (insufficient sample)

Patients' blood supplemented with betaine 50 μg/ml

| Patient ID | AspiTest | Collagen | ADP | TRAP |
|---|---|---|---|---|
| # 1 | 12 | 8 | 29 | 83 |
| # 2 | 6 | 3 | 6 | 30 |
| # 3 | 17 | 17 | 13 | 22 |
| # 4 | 97 | 76 | 89 | 93 |
| # 5 | 125 | 91 | 86 | 179 |
| # 6 | 87 | 75 | 89 | 120 |
| # 7 | 94 | 88 | 90 | 100 |
| # 8 | 77 | 46 | 115 | 127 |
| # 9 | 10 | 4 | 44 | 60 |
| # 10 | 22 | 3 | 1 | * |
| # 11 | 54 | 35 | 68 | 102 |
| # 12 | 18 | 21 | 46 | 79 |
| # 13 | 84 | 75 | 63 | 67 |
| # 14 | 94 | 91 | 77 | 103 |
| # 15 | 62 | 26 | 82 | 87 |
| # 16 | 29 | 34 | 57 | 84 |
| # 17 | 21 | 29 | * | 31 |
| # 18 | 49 | 21 | 83 | 103 |
| # 19 | 17 | 59 | 100 | 153 |
| # 20 | 5 | 25 | 81 | 152 |
| # 21 | 61 | 24 | 56 | 29 |
| # 22 | 121 | 42 | 173 | 155 |
| # 23 | 13 | 2 | 4 | 60 |
| # 24 | 75 | 30 | 70 | 141 |
| Mean | 52.08 | 38.54 | 66.17 | 93.91 |
| SD | 38.7 | 29.72 | 39.81 | 44.23 |

* not realised (insufficient sample)

Betaine effect

| | AspiTest | Collagen | ADP | TRAP |
|---|---|---|---|---|
| saline means | 62.54 | 48.46 | 71.78 | 108 |
| betaine means | 52.08 | 38.54 | 66.17 | 93.91 |
| Betaine additional | 17% | 20% | 8% | 13% |
| inhibition | | | | |
| P betaine vs. | 0.0109 | 0.0006 | 0.067 | 0.0392 |
| saline(Student) | | | | |

ASA and ASA/clopidogel non responders saline

| | AspiTest | Collagen | ADP | TRAP |
|---|---|---|---|---|
| # 4 | 119 | 71 | 80 | 119 |
| # 5 | 113 | 118 | 119 | 173 |
| # 6 | 116 | 77 | 86 | 104 |
| # 7 | 109 | 124 | 110 | 136 |
| # 13 | 100 | 79 | 71 | 104 |
| # 14 | 119 | 96 | 88 | 122 |
| # 18 | 80 | 52 | 87 | 121 |
| # 21 | 122 | 49 | 59 | 158 |
| # 22 | 150 | 37 | 143 | 176 |
| # 24 | 83 | 41 | 75 | 124 |
| mean | 111.1 | 74.4 | 91.8 | 133.7 |
| SD | 20.19 | 30.78 | 25.15 | 26.46 |

ASA and ASA/clopidogrel non responders betaine 50 μg/ml

| | AspiTest | Collagen | ADP | TRAP |
|---|---|---|---|---|
| # 4 | 97 | 76 | 89 | 93 |
| # 5 | 125 | 91 | 86 | 179 |
| # 6 | 87 | 75 | 89 | 120 |
| # 7 | 94 | 88 | 90 | 100 |
| # 13 | 84 | 75 | 63 | 67 |
| # 14 | 94 | 91 | 77 | 103 |
| # 18 | 49 | 21 | 83 | 103 |
| # 21 | 61 | 24 | 56 | 29 |
| # 22 | 121 | 42 | 173 | 155 |
| # 24 | 75 | 30 | 70 | 141 |
| mean | 88.7 | 61.3 | 87.6 | 109 |
| SD | 23.67 | 28.74 | 32.24 | 43.12 |

Betaine effect

| | AspiTest | Collagen | ADP | TRAP |
|---|---|---|---|---|
| Saline means | 111.1 | 74.4 | 91.8 | 133.7 |
| Betaine means | 88.7 | 61.3 | 87.6 | 109 |
| Additional | 20% | 17% | 5% | 19% |
| inhibition | | | | |
| P betaine vs. | 0.0043 | 0.0242 | 0.449 | 0.093 |
| saline(Student) | | | | |

### Comments

Betaine showed synergistic effects on different agonists used. These effects were significant (p<0.05) on AspiTest & collagen, confirming betaine synergistic effects with aspirin and/or clopidogrel.

Aspirin resistance ratio was as high as 40% in CAD patients. This corresponds to levels found by other workers, and now abundantly described in literature as a real public health problem.

Hence, such patients even administrated the 2 reference antiaggregant drugs (aspirin and clopidogrel) are not correctly protected as seen with their platelet function. Remarkably, betaine favourably and significantly affects their platelet function. This should afford better protection to cardiovascular patients in general and to aspirin resistant patients in particular.

### Example 2

### Nutritional Supplement

Beet molasses after desugarization processes are further submitted to one or more separation processes as to concentrate betaine and obtain betaine enriched molasses with 30% glycine betaine content. Such molasses are further dried and the obtained products grinded to powders having between 10 and 200 mesh sizes. The obtained powders are included in different dosage forms or edible products for human use.

### Example 3

Example 2 is repeated but with 50% glycine betaine content.

### Example 4

Example 2 is repeated but with 75% glycine betaine content.

### Example 5

Molasses or Products of examples 2, 3 or 4 are further submitted to one or more process reducing organic acids by 50% comparatively to the initial content of said organic acids.

### Example 6

Products of examples 2, 3, 4 or 5 are packaged in oral unit dosage form as sachets containing 2 to 20 grams. The said sachets having MVTR value inferior to 0.2 g/m².

### Example 7

Products of examples 2, 3, 4 or 5 are admixed with starch and compressed to produce tablets.

### Example 8

The powders and products of examples 2, 3, 4 or 5 are mixed and encapsulated to obtain titled granules.

### Example 9

The powders and products of examples 2, 3, 4 or 5 are mixed and encapsulated to obtain pills.

### Example 10

Gels using one or more of the products described in the application are realised and submitted optionally to one or more freezing and/or drying processes before being filled in one or more compartments selected from the group consisting of soft capsules, soft membranes and soft pills and theirs combinations.

### Example 11

20 patients attending Hospital cardiovascular consultation were enrolled for the study. Patients were all on 100 mg/day aspirin for their cardiovascular protection. Necessary ethical clearance was obtained from Hospital IRB.

Whole blood aggregation profile using Multiplate® instrument was assessed for each patient before and one hour after oral administration of 4 g of betaine. Blood was sampled on hirudin as anticoagulant.

Using the different test procedures available for the Multiplate® comprehensive information on platelet function and antiplatelet effects of betaine vs. basal values was assessed. Agonists were prepared in 1 ml solutions according to Multiplatc technical sheets and were used in the following volumes or concentrations: ASPI test: 20 μL, COL test: 20 μL, TRAP test: 20 μL, ADP test: 20 μL, RISTO test: 40 μL (1.2 mg/ml).

Results

Patients showing at basal AspiTest a response below 20 were considered as responders.

ASA responders

| | TRAP | Ristocetin | Collagen | ADP | AspiTest |
|---|---|---|---|---|---|
| Basal | | | | | |
| # 1 | 82 | 75 | 40 | 15 | 2 |
| # 3 | 164 | 12 | 5 | 89 | 6 |
| # 4 | 126 | 48 | 31 | 84 | 5 |
| # 5 | 146 | 84 | 28 | 95 | 5 |
| # 8 | 116 | 104 | 12 | 63 | 7 |
| # 10 | 36 | 80 | 9 | 3 | 3 |
| # 11 | 79 | 3 | 14 | 52 | 9 |
| # 13 | 65 | 15 | 13 | 32 | 9 |
| # 14 | 68 | 9 | 25 | 35 | 1 |
| # 18 | 110 | 36 | 6 | 75 | 4 |
| # 19 | 65 | 3 | 34 | 52 | 8 |
| # 20 | 70 | 33 | 51 | 54 | 7 |
| Mean | 93.92 | 41.83 | 22.33 | 54.08 | 5.5 |
| SD | 38.18 | 35.74 | 14.75 | 29.08 | 2.65 |
| After 4 g | | | | | |
| betaine | | | | | |
| # 1 | 64 | 39 | 11 | 2 | 11 |
| # 3 | 154 | 10 | 7 | 121 | 7 |
| # 4 | 115 | 42 | 29 | 64 | 4 |
| # 5 | 136 | 23 | 12 | 80 | 4 |
| # 8 | 131 | 35 | 23 | 93 | 9 |
| # 10 | 29 | 6 | 4 | 14 | 5 |
| # 11 | 80 | 3 | 10 | 51 | 1 |
| # 13 | 77 | 21 | 10 | 47 | 3 |
| # 14 | 67 | 14 | 3 | 44 | 6 |
| # 18 | 104 | 26 | 3 | 75 | 3 |
| # 19 | 69 | 9 | 30 | 45 | 9 |
| # 20 | 77 | 14 | 16 | 64 | 13 |
| Mean | 91.92 | 20.17 | 13.17 | 58.33 | 6.25 |
| SD | 36.28 | 13.12 | 9.5 | 32.54 | 3.62 |
| Difference | −2% | −52% | −41% | 8% | 14% |
| Vs. basal | | | | | |
| P Student | | 0.031 | 0.039 | | 0.6 |

Patients showing at basal A spiTest a response above 20 were considered as non responders.

ASA Poor Responders

| | TRAP | Ristocetin | Collagen | ADP | AspiTest |
|---|---|---|---|---|---|
| Basal | | | | | |
| # 2 | 90 | 33 | 53 | 102 | 35 |
| # 6 | 62 | 156 | 45 | 57 | 64 |
| # 7 | 147 | 115 | 38 | 56 | 81 |
| # 9 | 102 | 129 | 7 | 51 | 64 |
| # 12 | 66 | 66 | 15 | 61 | 62 |
| # 15 | 79 | 6 | 51 | 28 | 35 |
| # 16 | 88 | 27 | 67 | 59 | 22 |
| # 17 | 78 | 34 | 63 | 48 | 32 |
| Mean | 89 | 70.75 | 42.375 | 57.75 | 49.375 |
| SD | 26.77 | 55.45 | 21.53 | 20.7 | 20.88 |
| After 4 g | | | | | |
| betaine | | | | | |
| # 2 | 106 | 62 | 47 | 71 | 24 |
| # 6 | 66 | 78 | 48 | 53 | 74 |
| # 7 | 133 | 123 | 32 | 66 | 81 |
| # 9 | 103 | 81 | 18 | 60 | 69 |
| # 12 | 60 | 30 | 10 | 38 | 46 |
| # 15 | 66 | 2 | 58 | 32 | 27 |
| # 16 | 95 | 43 | 65 | 70 | 35 |
| # 17 | 106 | 11 | 57 | 40 | 52 |
| Mean | 91.875 | 53.75 | 41.875 | 53.75 | 51 |
| SD | 25.58 | 40.32 | 19.9 | 15.41 | 21.84 |
| Difference | 3% | −24% | −1% | −7% | 3% |
| Vs. basal | | | | | |
| P Student | | 0.22 | | 0.5 | |

Means of AUC aggregations of the 20 patients before and after 4 g betaine administration by oral route.

| | TRAP | Ristocetin | Collagen | ADP | AspiTest |
|---|---|---|---|---|---|
| Basal | | | | | |
| Mean | 91.95 | 53.4 | 30.35 | 55.55 | 23.05 |
| SD | 33.38 | 45.65 | 19.96 | 25.51 | 25.51 |
| After 4 g | | | | | |
| betaine | | | | | |
| Mean | 91.9 | 33.6 | 24.65 | 56.5 | 24.15 |
| SD | 31.67 | 31.36 | 20.16 | 26.56 | 26.25 |
| Difference | 0% | −37% | −19% | 2% | 5% |
| Vs. basal | | | | | |
| P Student | | 0.012 | 0.045 | | 0.57 |

### Comments

When assessed at basal with the Multiplate® aggregometer, 8 out of 20 patients showed at different levels to be aspirin resistant.

Betaine administration ameliorated significantly their platelet function on ristocetin aggregation (agglutination) and on collagen aggregation. This clearly shows that such aspirin poor responders or aspirin resistant patients are better protected with the betaine administration. Better results can be expected with betaine medicaments or betaine dietary supplements long term administrations.

1. A method of treating a human having a condition or being at risk for a condition selected from the group consisting of: (a) high levels of urinary and/or blood levels of 11-dehydro thromboxane B2 after being administered one or more antiplatelet compounds selected from the group consisting of aspirin, aspirin derivatives and combinations thereof; (b) high levels of platelet and/or blood aggregation after being administered one or more antiplatelet compounds selected from the group consisting of aspirin, aspirin derivatives and combinations thereof; (c) aspirin resistance; (d) poor response to aspirin; (e) resistance to at least two compounds selected from the group consisting of aspirin, clopidogrel, thienopyridines, and combinations thereof; (f) resistance to aspirin, clopidogrel, or thienopyridines, who suffers or who is at risk of suffering of cardiovascular diseases, cerebrovascular diseases, or combinations thereof, while being treated by one or more of said compounds; (g) cardiovascular diseases, cerebrovascular diseases, or combinations thereof who is aspirin resistant; (h) thromboembolic disorders, ischemic disorders, or combinations thereof, who is resistant to aspirin, clopidogrel, or thienopyridines; (i) thromboembolic disorders, in which an increase in thromboxane formation cannot be prevented by conventional dose rates of aspirin; (j) reperfusion damage/ischemia, myocardial infarction, thrombolysis, tumours, stroke or balloon dilatation; and (k) a disease requiring the administration of aspirin, in which an increase in thromboxane formation cannot be prevented by conventional dose rates of aspirin, said method comprising:
administering to said human a therapeutically effective amount of a betaine, a betaine enriched molasses, or a combination thereof.

2. The method of claim 1, wherein said condition is (a), and wherein the urine level of 11-dehydro thromboxane B2 is greater than 15 ng/mmol creatinine.

3. The method of claim 1, wherein said condition is (b), and wherein blood aggregation is realised using as an agonist arachidonic acid, collagen, or both.

4. The method of claim 3, wherein the threshold concentration for the agonist is 1 μg/ml for collagen and 0.5 mM for arachidonic acid.

5. The method of claim 1, wherein said condition is (b), and wherein the levels of platelet or blood aggregation of said human are inferior by less than 20% compared to the levels of platelet or blood aggregation of said human prior to being administered one or more antiplatelet compounds selected from the group consisting of aspirin, aspirin derivatives and combinations thereof.

6. The method of claim 1, wherein said condition is (c), and wherein said aspirin resistance is decreased by at least 10%.

7. The method of claim 1, wherein said condition is (a), and wherein said high levels of urinary and/or blood levels of 11-dehydro thromboxane B2 are decreased by at least 10%.

8. The method of claim 1, wherein said betaine comprises an effective amount of glycine betaine for rendering said patient substantially no more resistant to aspirin, clopidogrel and thienopyridines.

9. The method of claim 1, wherein said condition is (k), said betaine is glycine betaine or glycine betaine enriched molasses, and wherein said aspirin resistance is decreased by at least 10%

10. A betaine enriched molasses comprising between 10% and 95% of betaine on a dry matter basis for the fabrication of a nutritional product.

11. The betaine enriched molasses according to claim 10, said nutritional product being effective for achieving one or more health and/or therapeutical purpose selected from the group consisting of: improve athletic performance, improve haemostasis, sustain healthy haemostasis, favour healthy haemostasis, improve favour haemostasis, sustain haemostasis, ameliorate haemostasis, reinforce haemostasis, improve cardiovascular function, sustain healthy cardiovascular function, favour healthy cardiovascular function, favour cardiovascular function, sustain cardiovascular function, ameliorate cardiovascular function, reinforce cardiovascular function, improve blood circulation, favour healthy blood circulation, favour blood circulation, sustain healthy blood circulation, sustain blood circulation, ameliorate blood circulation, reinforce blood circulation, improve blood microcirculation, favour blood microcirculation, sustain healthy blood microcirculation, sustain blood microcirculation, ameliorate blood microcirculation, reinforce blood microcirculation, improve platelet function, improve healthy platelet function, favour healthy platelet function, favour platelet function, sustain healthy platelet function, sustain platelet function, ameliorate platelet function, pacify platelet, favour platelet pacification, sustain platelet pacification, reinforce platelet pacification, reinforce platelet function, favour healthy sensitivity to aspirin, improve sensitivity to aspirin, favour sensitivity to aspirin, sustain healthy sensitivity to aspirin, sustain sensitivity to aspirin, ameliorate sensitivity to aspirin, reinforce sensitivity to aspirin, favour healthy sensitivity to clopidogrel, improve sensitivity to clopidogrel, favour sensitivity to clopidogrel, sustain healthy sensitivity to clopidogrel, improve sensitivity to clopidogrel, sustain sensitivity to clopidogrel, ameliorate sensitivity to clopidogrel, reinforce sensitivity to clopidogrel, favour healthy sensitivity to thienopyridines, improve sensitivity to thienopyridines, favour sensitivity to thienopyridines, sustain healthy sensitivity to thienopyridines, improve sensitivity to thienopyridines, sustain sensitivity to thienopyridines, ameliorate sensitivity to thienopyridines, reinforce sensitivity to thienopyridines, reduce the risk of aspirin resistance, reduce the risk of clopidogrel resistance, reduce the risk of thienopyridines resistance, ameliorate aspirin resistance, ameliorate clopidogrel resistance, ameliorate thienopyridines resistance, reduce the risk of diabetes, ameliorate diabetes, improve glycemia in diabetic states, ameliorate glycemia in diabetic states, ameliorate insulin resistance in diabetic states, favour healthy insulin sensitivity, improve insulin sensitivity, favour insulin sensitivity, sustain healthy insulin sensitivity, sustain insulin sensitivity, ameliorate insulin sensitivity, reinforce insulin sensitivity, favour healthy glycemia, favour normal glycemia, sustain healthy glycemia, sustain normal glycemia, ameliorate glycemia, reinforce healthy glycemia, reduce the risk of high glycemia, improve glucose utilisation, ameliorate glucose utilisation, ameliorate glucose utilisation by the body, favour healthy legs circulation, favour legs circulation, sustain healthy legs circulation, improve legs circulation, sustain legs circulation, ameliorate legs circulation, reinforce legs circulation, favour healthy erectile function, improve erectile function, favour erectile function, sustain healthy erectile function, sustain erectile function, ameliorate erectile function, reinforce erectile function, relieve the haemorrhoids crisis, relieve heavy legs, reduce the risk of inflammation, reduce the risk of cancer, reduce the risk of Alzheimer Disease, ameliorate vision; and the mixtures of such health purposes.

12. The betaine enriched molasses according to claim 10, wherein said molasses comprises between 20% and 80% of betaine on a dry matter basis.

13. The betaine enriched molasses according to claim 10, wherein said nutritional product is effective for treating or preventing aspirin resistance in a patient in need.

14. The betaine enriched molasses according to claim 10, wherein said nutritional product is effective for treating or preventing clopidogrel resistance and/or thienopyridines resistance in a patient in need.

15. A betaine enriched molasses according to claim 10, further comprising 5 to 65% sugar, 1 to 40% essential amino acids, 1 to 30% nitrates, and 1 to 40% organic acids.

16. A betaine enriched molasses according to claim 15, wherein said essential amino acids are selected from the group consisting of one or more lysine, methionine, cystine, tryptophan, threonine and theirs mixtures.

17. A betaine enriched molasses according to claim 15, wherein said organic acids are selected from the group consisting of one or more lactic acid, malic acid, acetic acid, oxalic acid, glutammic acid, citric acid, succinic acid and theirs mixtures.

18. A betaine enriched molasses according to claim 10, wherein said nutritional product is sealed in a unit dosage form which is provided with a moisture barrier property defined by an increase of weight of the compositions of less than 1% after storage of the unit dosage form in scaled condition in an environment with a temperature of 38° C. and a relative humidity of 90% during 30 days.

19. A betaine enriched molasses according to claim 18, wherein the unit dosage form is selected from the group consisting of sachets, pouches, blisters and bags, wherein the pharmaceutical unit dosage form is provided with moisture barrier property defined by an MVTR value inferior to 0.2 g/m² at a temperature of 38° C. and at 90% relative humidity during 24 hours.

20. A betaine enriched molasses according to claim 10, wherein said nutritional product is in an oral dosage form selected from the group consisting of drop pills, spray solution, pellets, pills, granules, gels, capsules, tablets, powders, gel soft capsules, gels soft pills and oral liquids.

21. A process for obtaining a betaine enriched molasses according to claim 10 for the fabrication of a medicament and/or nutritional product comprising:
providing desugarized beet molasses, cane molasses, or a combination thereof; and
subjecting said desugarized molasses to a separation process to yield betaine enriched molasses.

22. The process of claim 21, further comprising submitting said betaine enriched molasses to one or more drying processes selected from the group consisting of drying processes, spray-drying, tumble-drying, lyophilisation processes, freeze-drying processes, and combinations thereof.

23. The process of claim 22, further comprising submitting the dried betaine enriched molasses to one or more of another process selected from the group consisting of powdering, micronizing, grinding, crushing, granulating, and combinations thereof.
